(19) United States
(12) Patent Application Publication
(10) Pub. No.: US 2022/0022806 A1
(43) Pub. Date: Jan. 27, 2022

(54) SYSTEM AND METHOD TO DETECT AND TRACK CELLULAR CHANGES IN HEALTHY BREAST TISSUE ASSOCIATED WITH BREAST DENSITY, MENOPAUSAL STATUS AND AGE

(71) Applicant: Translational Research Institute Pty Ltd as trustee for Translational Research Institute Trust, Woolloongabba (AU)

(72) Inventors: Carolyn Mountford, Ryde (AU); Gorane Santamaria, Bowen Hills (AU); Peter Malycha, St Georges (AU); Natali Naude, Ninderry (AU)

(73) Assignee: Translational Research Institute Pty Ltd as trustee for Translational Research Institute Trust, Woolloongabba (AU)

(21) Appl. No.: 17/383,003

(22) Filed: Jul. 22, 2021

Related U.S. Application Data
(60) Provisional application No. 63/055,140, filed on Jul. 22, 2020.

(51) Int. Cl. A61B 5/00; A61B 5/055; A61B 5/1468; G16H 50/70

(52) U.S. Cl. CPC A61B 5/4312; A61B 5/0075; G16H 50/70; A61B 5/1468; A61B 5/055

(57) ABSTRACT

A method and system provides the ability to detect density of a women's breast by obtaining the concentration of at least one selected biochemical in the breast using a spectrometer, and comparing the concentration obtained with reference measurements which correlate breast density with concentration of the at least one selected biochemical.

### CROSS-REFERENCE TO RELATED APPLICATIONS

This application claims priority to, and incorporates by reference, U.S. Provisional Application Ser. No. 63/055,140, filed Jul. 22, 2020.

### TECHNICAL FIELD

The present invention is directed to a system and method to detect and track cellular changes in breast tissues of women associated with breast density, age and menopause, which are indicative of increased risk to breast cancer.

### BACKGROUND OF THE INVENTION

Throughout this application, references are cited and are listed at the end of the specification. These references are incorporated by reference herein.

Epidemiological studies have shown breast density is an independent risk factor for breast cancer [1-3]. Studies suggest that increased breast density may make a woman 4 to 6-fold more likely to develop breast cancer [4]. The relative risk associated with high breast density may be greater than a family history of breast cancer. Only age and BRCA mutation status are associated with a higher risk [5]. The most widespread method to qualitatively assess breast density is the Breast Imaging Reporting and Data System (BI-RADS) classification. BI-RADS evaluates parenchymal patterns and distributions, classifying the tissue density into four categories [6].

Magnetic resonance (MR) imaging (MRI) is a commonly used tool to identify breast cancer but it is unable to distinguish cellular, chemical, or metabolic changes that occur before a cancer develops. We have used in vivo 1D and 2D MR spectroscopy to evaluate these changes. We have found breast tissue is difficult to evaluate using conventional methods using one dimensional in vivo (1D) MR spectroscopy. The lipid signal is so intense that it masks the details of many of the chemical changes taking place. This issue has been overcome by the use of in vivo two-dimensional (2D) MR spectroscopy where the chemicals can be unambiguously assigned in a second magnetic frequency [7, 8].

In vivo 2D Correlated SpectroscopY (2D COSY) was successfully used to evaluate breast tissue chemistry in women carrying the BRCA1 or BRCA2 gene mutations [7]. The neutral lipid changes recorded differed between the BRCA1, BRCA2 and healthy cohorts. However, the signal to noise was not adequate to study any changes in metabolites in women with these two genetic mutations and the healthy cohort.

There is a need to evaluate women with presumably healthy breast in the general population to determine molecular changes due to age, breast density or menopausal status in a non-invasive manner. The capacity to non-invasively monitor quantitative changes at a molecular level, in an apparently healthy breast, would be a major advance in healthcare for women.

### SUMMARY OF THE INVENTION

The invention provides the capacity to non-invasively monitor changes at a molecular level, in an apparently healthy breast, and provides a major advance in healthcare for women. Using state of the art magnetic resonance (MR) scanners, and new age coil technology, chemical changes occurring with breast density and menopausal status can be monitored, and compared to a reference database of chemical values of selected biochemical from those of healthy breast women having relatively low breast density, and those of women having relatively high breast density, to determine the breast density of a subject and the risk of breast cancer. Increases in cholesterol, triglycerides, unsaturated lipid content and an array of metabolites indicate the presence of high density breast tissue compared to low density tissue. The high density breast tissue is associated with increased risk of breast cancer, even though it is not necessarily an indication of breast cancer. Data from women can be categorized into four groups: low density pre-menopausal, low density post-menopausal, high density pre-menopausal and high density post-menopausal. Among these four groups, a gradual increase in chemical activity through this series was observed. The MR characteristics of breast tissue in post-menopausal women with high density tissue is comparable with stimulated cells that do not proliferate and suggest a link to inflammation. This new capability provides an objective estimation of breast cancer risk and the capacity to monitor the healthy breast in a way not previously possible. The new capability can be done non-invasively, in vivo, using a spectrometer and avoiding the use of contrast agent needed for an MRI.

We also have found that using data mining techniques [9], wherein each of the 4096 data points are evaluated, can be used. The frequencies are identified to distinguish between categories, and the clinical information pertaining to breast density, age and menopausal status can be determined. Two of these three frequencies are outside the spectral envelope at 0.4, 0.7 and 5.2 ppm. The third has not yet been chosen. These are from cholesterol, cholesterol and the olefinic representing the C═C in the fatty acyl chains. None of this has been seen before in vivo.

The method identifies biomarkers using statistical classification algorithms with a high rate of diagnostic accuracy and classification algorithm to identify spectral changes that distinguish control subjects according to breast density, age and menopausal status.

More recently advances in MR hardware, including magnet stability, coil technology and the capacity for radiographers to operate the scanner with precision in spectroscopy mode, has resulted in much improved signal to noise ratios which allows us to assess changes in lipids, metabolites and carbohydrates.

The invention provides a method for enabling detection of breast density of a subject, comprising: obtaining spectral data, using a spectroscopy device, of a breast of a subject, and processing the spectral data with a processor to obtain a measurement of the concentration of at least one selected biochemical whose concentration varies with breast density, to enable a comparison of the measurement of the concentration with reference measurements of the concentration of the selected biochemical of subjects having varying known levels of breast tissue density correlated with the concentration of the selected biochemical, to enable a determination of the breast density of the subject by reference to the concentration of the selected biochemical.

The selected biochemical may be at least one of cholesterol (sterol and methyl), triglycerides, unsaturated fatty acyl chains or at least one of selected metabolites. The at least one of the selected metabolites may be choline, tyrosine, glycerophosphocholine, glutamine/glutamate, ethanolamine, composite choline, phosphocholine, taurine, glucose, scyllo-inositol, glucose, myo-inositol, creatine, GPC, aspartate, phosphocreatine, composite choline or myo-inositol. The reference measurements may be those of pre-menopausal women and post-menopausal women in separate groups, and wherein the spectral data of the subject are compared to the reference measurements of the relevant group depending on whether the subject is pre-menopausal or post-menopausal.

The invention provides a system for enabling detection of breast density of a subject, comprising: a spectrometer for obtaining spectral data of a breast of a subject, and a processor for processing the spectral data to obtain a measurement of the concentration of at least one selected biochemical whose concentration varies with breast density to enable a comparison of the measurement with reference measurements of the concentration of the selected biochemical of subjects known to have varying known levels of breast tissue density correlated with the concentration of the selected biochemical, to enable a determination of the breast density of the subject by reference to the concentration of the selected biochemical.

The invention provides a method of making a breast density detection system for enabling a determination of breast density of a subject using the concentration of at least one selected biochemical in the subject's breast tissue whose concentration varies with breast density, comprising: using a magnetic resonance imaging device to obtain magnetic resonance images of a plurality of breasts of women having different breast densities; using a spectrometer to obtain spectral data from the plurality of the women's breasts to obtain the concentration of at least one selected biochemical in the plurality of the breasts, wherein the concentration of the selected bio-chemical varies with the breast density; and using a processor to correlate the breast density with the concentration of the selected biochemical to obtain a reference system of reference measurements which correlates breast density with the concentration of the selected biochemical, whereby the breast density of a subject can be determined by obtaining the spectral data and the concentration of the selected biochemical.

The invention provides a method of using a breast density detection system to determine the breast density of the subject, the system having been obtained by: using a magnetic resonance imaging device to obtain magnetic resonance images of a plurality of breasts of women having different breast densities; using a spectrometer to obtain spectral data from the plurality of breasts to obtain the concentration of at least one selected biochemical in the plurality of the breasts which concentration varies with the breast density; and using a processor to correlate the breast density with the concentration of the selected biochemical to obtain a reference system of reference measurements which correlates breast density with the concentration of the selected biochemical, wherein the method of using comprises obtaining spectral data of the subject's breast with a spectrometer, and using a processor to determine the concentration of the selected biochemical, and to determine the breast density by reference to the breast density which correlates with the concentration of the selected biochemical.

### BRIEF DESCRIPTION OF THE DRAWINGS

FIG. 1A shows typical localized COSY spectra with the cross peaks assigned (A-G′) as per FIG. 1B. FIG. 1A-(a) shows a low dense tissue and pre-menopausal status, FIG. 1A-(b) shows a low dense tissue and post-menopausal, FIG. 1A-(c) shows a high dense tissue and pre-menopausal and FIG. 1A-(d) shows high dense tissue and post-menopausal. There was clear increase in intensity for cross peaks G and G′, cross peak D and cross peak C, from top to bottom.

FIG. 1B shows a Triglyceride molecule with the cross peaks labelled (A-G′).

FIGS. 2a, 2b, 2c and 2d show 3D plots and contour plots of the expanded region F2/F1: 3.00 ppm to 3.90 ppm. Some of the metabolites resonating in this region across the four categories are denoted: FIG. 2a shows Low density tissue and pre-menopausal status, FIG. 2b shows Low density and post-menopausal, FIG. 2c shows High density tissue and pre-menopausal, FIG. 2d shows High density tissue and post-menopausal. 3D plots illustrate intensity of each of the metabolites. Contour plots demonstrate the frequencies of each diagonal resonance. The magnification in FIGS. 2c and 2d are three times that of 2a and 2b. Tentative assignments of Cho: Choline; Cr: Creatine; EA: Ethanolamine; Gly: Glycerol; GPC: Glycerophosphocholine; Gcn: Glycine; Glc: Glucose; Gln: Glutamine; Glu: Glutamate; His: Histidine; m-Ins: Myo-inositol; PC: Phosphocholine; PCr: Phosphocreatine; s-Ins: scyllo-Inositol; Tau: Taurine; Thr: Threonine; Tyr: Tyrosine.

FIG. 3A shows bar graphs which display the average peak volumes of cholesterol.

FIG. 3B (top insert) shows the lipids across the four categories.

FIG. 3C shows the metabolites are shown across the four categories. Lipids and metabolites assignments are as follows: (LIPIDS) cross peak C (2.02, 5.31 ppm); cross peak D (2.75, 5.31 ppm); —CH₂—(C═O)—O-/cross peak G′ (4.10, 4.25 ppm); composite from —CH₂—CH₂—(C═O)—O-/methine protons alkyl side chain cholesterol (1.59, 1.59 ppm); —HC═CH— (5.31, 5.31 ppm). (METABOLITES) choline/phosphocholine (3.18-3.26); taurine, glucose (3.25 ppm); myo-inositol (3.27 ppm); choline, myo-inositol (3.45-3.55 ppm); GPC/glutamine (3.67-3.73 ppm); glycerol, alanine ((3.76-3.80 ppm); creatine, GPC, aspartate, phosphocreatine (3.85-3.95 ppm).

### DETAILED DESCRIPTION OF THE PREFERRED EMBODIMENT

A preferred embodiment of the invention will be provided, but the invention is not limited to this embodiment.

As used herein, the term “spectrometer” means a spectrometer, or an MRI or MR scanner operating in a spectroscopy mode to obtain spectral data in vivo.

Recent developments in 3 Tesla MR scanner and coil technology have made it possible to evaluate the tissue chemistry of the human body with improved spectral resolution and signal to noise. This has only been possible previously using cell lines and biopsies at higher magnetic field strengths [10]. The capacity to study the healthy state, ex vivo or in vitro with cultures, remains extremely difficult. The capacity to do so in vivo generates a range of previously unavailable information in other organs[11].

We have now recorded in vivo, chemical changes that take place in the healthy breast tissue of women with an average risk of breast cancer and correlated these findings with their breast density and age/menopausal status. The new information provides a clearer understanding of why breast density is an independent risk factor for breast cancer.

The changes in tissue chemistry, recorded in this series, include neutral lipids and metabolites. Each needs to be considered separately as neutral lipids, triglycerides and cholesterol have a natural affinity. They create neutral droplets or domains like those found in serum lipoproteins. Articles published from the 1980s are informative as to how these neutral lipids behave and their spectral characteristics [12].

The resonance at 0.70 ppm, has previously been assigned to cholesterol C18 [12], and shown to have rapid molecular motion, even when the rest of the molecule is relatively restricted. The chemical affinity of neutral lipids does not alter whether they are in a test tube, cell, or organ. They create neutral droplets or domains like those found in serum lipoproteins.

There are two possible explanations for the location of these increased levels of neutral lipids in the high density post-menopausal breast tissue. The first is in the cytoplasm where they would provide the lipid pool for rapid doubling of cells when needed. The second is in the plasma membranes of activated or stimulated cells, such as macrophages or other inflammatory cells.

A model was proposed whereby neutral lipid domains are intercalated with the bilayer lipid of the plasma membrane of cells which are activated, stimulated or transformed [13]. This model was contentious [14] but verified many years later [15]. As the triglyceride and cholesterol ester levels increase, the cholesterol becomes more mobile and can be measured. King et al [16] used 2D COSY to study murine macrophages and found that proliferation is not a prerequisite for acquisition of an ‘activated’ high resolution spectrum in cell models. Dense breast tissue, in post-menopausal women, has also been associated with a pro-inflammatory cytokines and significantly increased number of inflammatory cells [17]. Increased levels of pro-inflammatory cytokines have also been shown to stimulate triglyceride synthesis, cholesterol accumulation and de novo lipogenesis [18] in the post-menopausal women with high dense breast tissue. Inflammation is considered as one of the hallmarks of cancer initiation and progression [19].

The resonances on the diagonal are still composites. With the next improvements in scanner capabilities, and further increases in signal to noise ratio, they will also likely be seen as cross peaks and thus assigned unambiguously. From current tentative assignments those metabolites increased through the series include glucose, choline, glycerol, myo-inositol, taurine and glutamine/glutamate. These changes are consistent with reports in the literature.

Morris et al [20] reported a decrease in glucose metabolism via the tricarboxylic acid cycle and oxygen consumption in high density tissue collagen matrices, using an in vitro 3D model. Similarly others [21] reported that glucose metabolism significantly increased in high density tissue, but was not affected by menopausal status.

Cytokines are also reported to be involved in the development of abnormal glucose metabolism [18].

We recorded a steady increase in myo-inositol throughout this series. Myo-inositol has been shown to modulate inflammatory, oxidative, endocrine and metabolic pathways [22]. Likewise, this metabolite also regulates the transforming growth factor β-activity which induces collagen synthesis and modulates the matrix-degrading metalloproteinases and their inhibitors in the breast tissue [22]. In parallel, taurine has been reported to be an anti-oxidant that exerts antineoplastic effects through downregulation of angiogenesis and suppressing cell proliferation [23]. Thus, some of these changes recorded in the healthy breast are protective. In summary our results show that post-menopausal women with high density exhibit breast tissue chemistry similar to that observed in activated cells with increase in cholesterol, triglycerides and unsaturation of fatty acyl chains. The metabolites that are recorded to increase are consistent with inflammatory, oxidative and metabolic pathways being activated. Some metabolites are also part of protective mechanisms.

Inflammation is considered as one of the hallmarks of cancer initiation and progression [19]. The results presented here enable a detection of high density breast tissue in women who would be more likely to develop cancer.

From a clinical perspective, the capacity to monitor these tissue changes in healthy breast tissue without the use of a contrast medium could be an important step towards managing the health and risk of women as they age. Women at elevated risk for breast cancer could now have this evaluation undertaken at the same time as a routine MRI. Automated processes for data mining and classifiers can be developed to automate the process.

In conclusion, the in vivo MR spectroscopy both 1D and 2D COSY protocols, using a state-of-the-art clinical MR scanner can record changes to tissue chemistry as a consequence of breast density, menopausal status and age. Four categories were identified with increasing neutral lipid and metabolite activity in the following order low density pre-menopausal, low density post-menopausal, high density pre-menopausal and high density post-menopausal. Markers of inflammation gradually appearing through this series are consistent with the literature. This technology now paves the way for the healthy breast to be evaluated in conjunction with other risk criteria for cancer. It also allows each woman to be her own control during the aging process.

### Materials and Methods

### Patient Cohort and Inclusion Criteria

A cross-sectional study was undertaken with prospective data collection at three hospitals. Sixty-five healthy female volunteers at low risk of developing breast cancer according to National Institute for Health and Care Excellence (NICE) guidelines [24] were consecutively recruited from the wider community. Under the NICE guidelines, low risk is deemed as the general population lifetime risk below 17% and is assigned to a person who fulfils any of the following criteria: a) no family history of breast cancer, b) one first-degree relative diagnosed with breast cancer above age 40, c) one first degree relative and one second-degree relative with breast cancer with onset at any age, or d) two first- or second-degree relatives diagnosed with breast cancer above age 50 on different sides of the family.

The lifetime risk was calculated for each patient according to the International Breast Cancer Intervention Study (IBIS) score using the Tyrer-Kuzick model [25].

### MR Imaging

The participants underwent non-contrast MR imaging of the breast and in vivo MR 2D COSY between days 6 and 14 (follicular phase) of the menstrual cycle, where relevant. The data were collected on a 3T Prisma or a 3T Vida scanner (Siemens AG, Erlangen, Germany) using either an 18-channel (Siemens AG, Erlangen, Germany) or a 16-channel (RAPID Biomedical, Germany) breast coil.

Breast MRI consisted of: a) localizer sequence (repetition time (TR) 6 ms, echo time (TE) 2.61 ms, slice thickness 7 mm, field of view (FoV) 400 mm); b) axial T1-weighted 3D flash (TR 5.43 ms, TE 2.46 ms, flip angle 20°, slice thickness 2 mm, FoV 30 mm, matrix 448×448 mm); c) axial T2-weighted TSE sequence (TR 4280 ms, TE 97 ms, slice thickness 2 mm, FoV 300 mm, matrix 448×448 mm). Where possible, diffusion-weighted sequence (TR 5940, TE1 58 ms, TE2, 99 ms, slice thickness 4 mm, FoV 340 mm, matrix 274×274 mm) was also performed.

Two radiologists (20 years and 10 years' experience) undertook the BI-RADS assessment using a T2-weighted sequence. Breast density categories were type a (fatty breast tissue), type b (scattered density), type c (heterogeneous density) and type d (extremely dense breast tissue) [6].

### One-Dimensional Spectroscopy:

1. PRESS (TR: 2000 ms; TE: 33 ms; 16 Averages; Weak water suppression; Bandwidth 1500 Hz; Delta frequency −1.5 Hz; Flip angle 90 degrees). An automatic pre-scan is used to adjust frequency, transmitter voltage, water suppression and shimming. Data is collected with and without water suppression. Prior to acquisition, automated shimming is performed. Spectral line widths are considered acceptable if below 50 Hz. If necessary, voxel location is modified and shimming repeated. Following shimming, spectroscopy is performed. Scan time totals 55 seconds.

2. PRESS (TR: 2000 ms; TE: 135 ms; 64 Averages; Weak water suppression; Bandwidth 1500 Hz; Delta frequency: −1.5 Hz; Flip angle 90 degrees). Voxel is acquired from the same location as the TE 33 ms acquisition, and the same shim settings are used. An automatic pre-scan is used to adjust frequency, transmitter voltage, water suppression and shimming. Data is collected with and without water suppression. Scan time totals 3 minutes.

### Two-Dimensional MR Correlated Spectroscopy

A 3D T1-weighted sequence was used to position an 8 mm3 (20×20×20 mm3) voxel in the mid aspect of the left breast. The breast was positioned as close as possible to the magnet isocenter to minimise B0 inhomogeneity, thereby improving the quality of the shim. It included a region representative of the overall BD and avoided the para-areolar region, cystic regions, and large blood vessels. Localized shimming was performed using the automatic B0-field mapping technique Siemens auto-shimming algorithm [26], followed by manual adjustment of zero order shim gradients to achieve a width of the water peak at half maximum of ≤65 Hz. The 2D COSY sequence parameters were TR 2000 ms, TE initial of 30 ms, 96 increments, 6 averages pe₂r increment, bandwidth 2000 Hz, T1 increment 0.8 ms, vector size of 1024 points and RF offset frequency set on 3.2 ppm. ‘WET’ water suppression [27] was applied prior to acquisition. Processing was undertaken as reported [7]. Cross peak and diagonal peak volumes were measured using the Felix software (Accelrys. 2007) with the (CH₂)n diagonal peak at 1.30 ppm on the diagonal as the internal chemical shift reference.

### Statistical Analysis

Age, BI-RADS category of BD, menopausal status, BMI, IBIS risk score as well as measured volume of various lipid diagonal peaks and cross peaks, metabolites and cholesterol were collected for each participant. Family history of breast cancer including age of onset and whether disease was bilateral was recorded. Chi-squared or Fisher exact test, where appropriate, were used to compare categorical variables. Mean comparison between groups was performed using Mann-Whitney non-parametric test. Inter-observer variability was assessed by kappa statistics for qualitative data. A two-sided p-value of <0.05 was considered statistically significant. Statistical analysis was undertaken using IBM SPSS Statistics 25.0 (IBM, Armonk, N.Y.).

### Results

### Clinical Features

The demographics of this cohort and apparent diffusion coefficient values from breast density categories are listed in Table 1. Sixty nine percent (45/65) of participants were pre-menopausal and the remaining 31% (20/65) post-menopausal. The breast density distribution in this cohort were made up of 14% (9/65) type a, 39% (25/65) type b, 32% (21/65) type c and 15% (10/65) type d. The lower density category types a and b have been combined as have the higher density category types c and d. The inter-observer variability for the breast density assessment was excellent with a kappa coefficient=0.819 (p<0.001). Participants with high breast density were significantly younger than those in the low density category (p=0.004) and 84% were pre-menopausal (p=0.011).

Sixty nine percent of the women had no family history of breast cancer and 31% had one first-degree relative with breast cancer above age forty. There was no significant association between breast density and family history of breast cancer (p=0.663).

The apparent diffusion coefficient value was higher in the sampled voxel in high dense tissue than in low dense tissue (p<0.001).

### In Vivo MR Spectroscopy of Healthy Human Breast Tissue

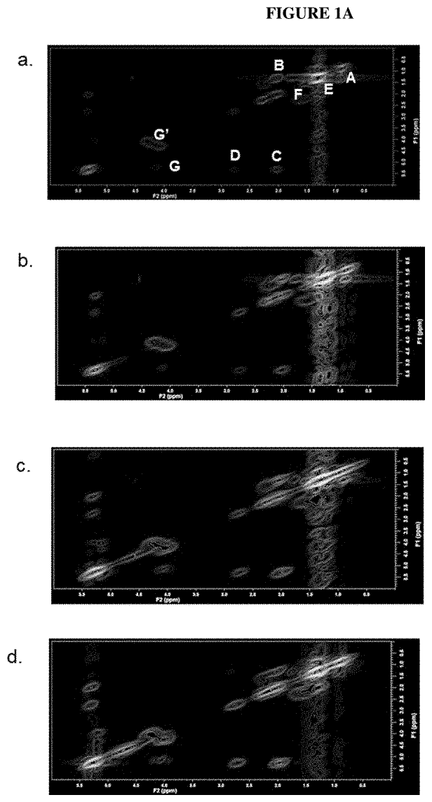

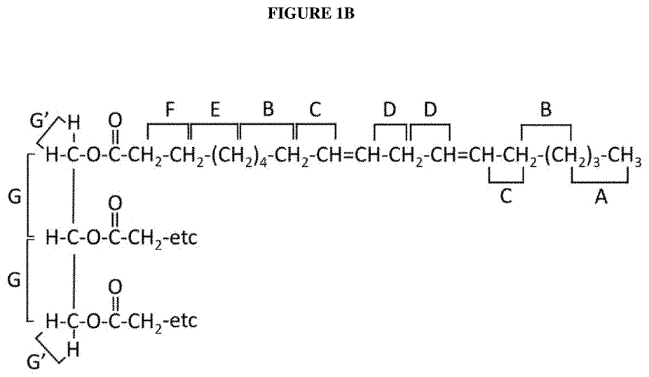

A comparison has been made between four categories of women viz. low density pre-menopausal, low density post-menopausal, high density pre-menopausal and high density post-menopausal. Typical 2D MR Spectra from each group are shown in FIG. 1A. The lipid assignments are as previously reported [7] and the off-diagonal cross peaks are labelled as A-G′, indicating the spin-spin coupling between protons on adjacent carbon atoms. The connectivity corresponding to each cross peak (A-G′) from the triglyceride molecule is shown in FIG. 1B. Triglyceride possesses a unique cross peak G′ at 4.25 ppm resulting from the geminal protons of carbons 1 and 3 of the glycerol backbone (FIG. 1B). Cross peak G arises from the methylene-methine coupling on the glycerol backbone of triglyceride and is seen as two clear cross peaks in these breast spectra (FIG. 1A), which has not been reported previously in vivo from the human breast. There is a visual increase in intensity for cross peaks G and G′, cross peak C (F2: 2.02, F1: 5.31 ppm) and cross peak D (F2: 2.75, F1: 5.31 ppm) throughout the four categories (FIG. 1A).

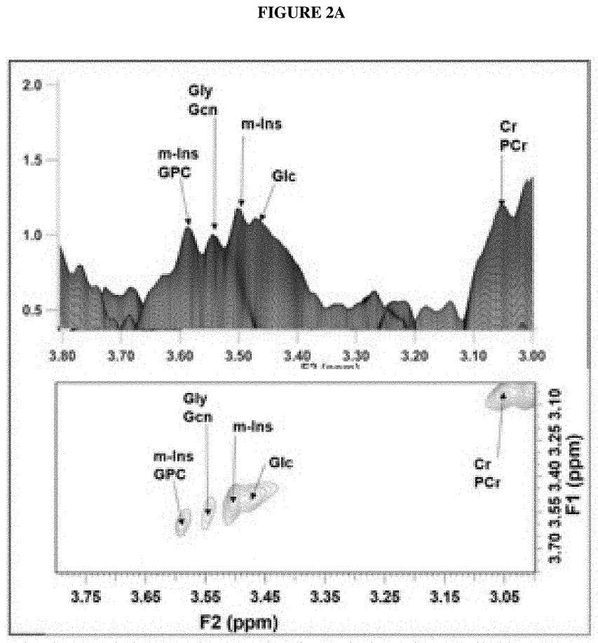

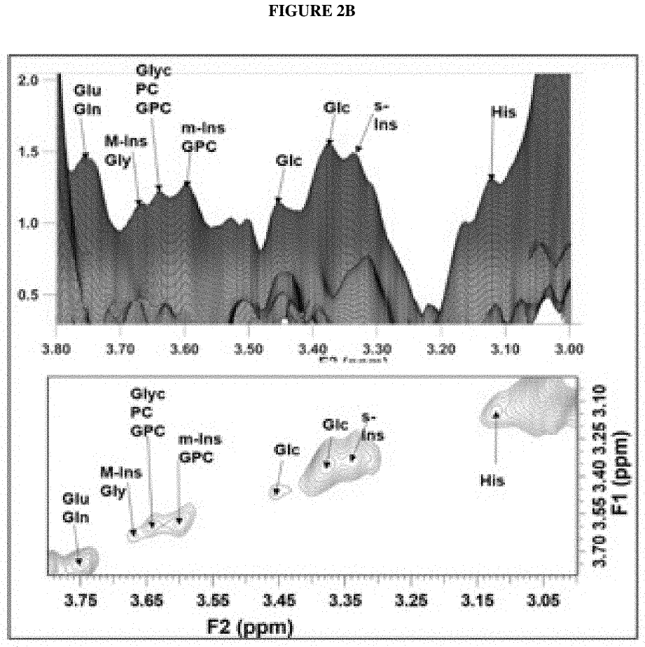

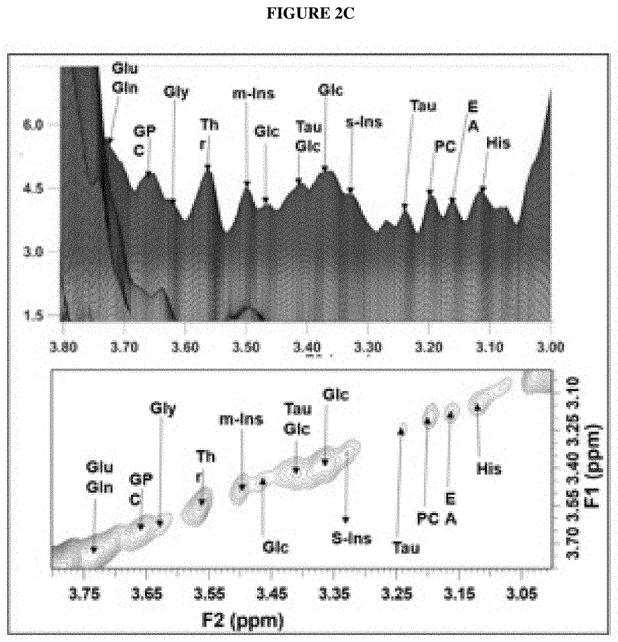

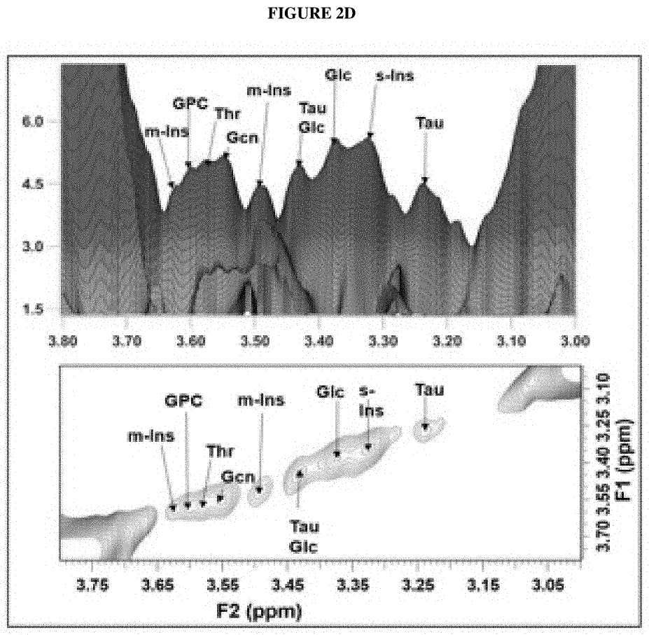

The diagonal region 3.00 ppm to 3.90 ppm from each of these 2D spectra are magnified and shown in FIG. 2. The spectra are displayed in two ways; as a 3D plot and a contour plot. The former shows the spectral frequencies clearly and the latter, the range of intensities. A clear visual increase is observed in the number of metabolites that are available for inspection in this series.

### Chemical Differences Between Categories

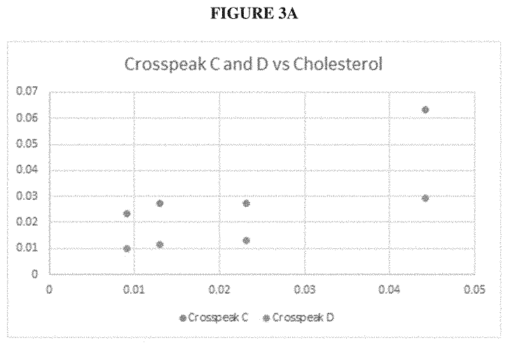

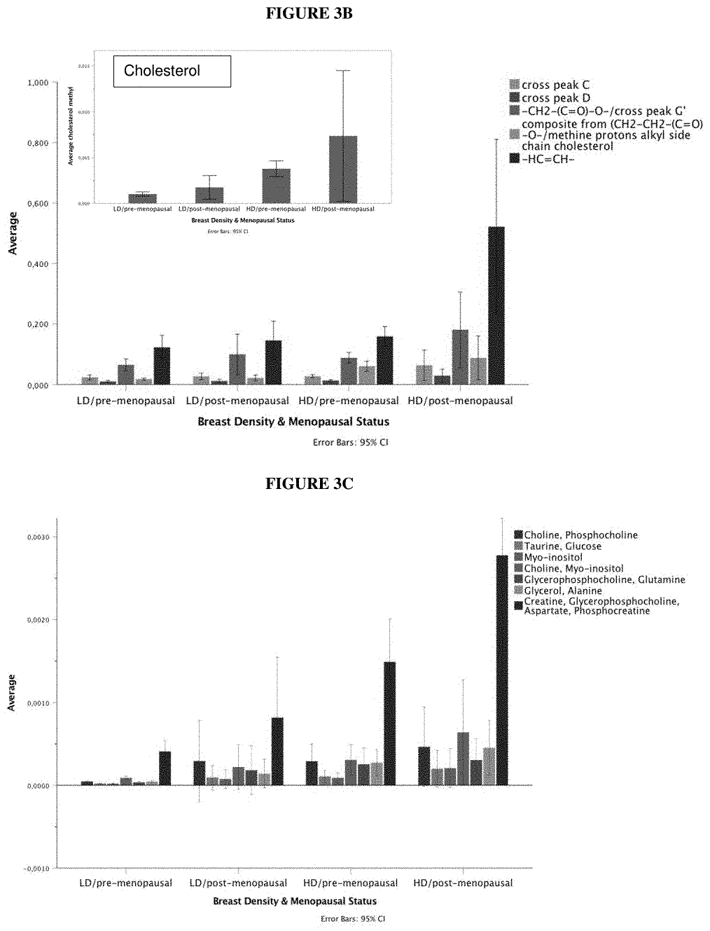

The cross peak and diagonal peak volumes from the 2D COSY were measured for each category and the results summarised in Table 2 and FIG. 3. We compared the effect of breast density on tissue chemistry according to menopausal status and age.

### Pre-Menopausal Participants

Pre-menopausal women with high dense tissue showed a 105% increase in the cholesterol methyl (F2:0.70, F1:0.70 ppm) of 156% (p<0.001), cholesterol sterol (F2:0.40, F1:0.40 ppm) of 105% (p=0.002) and the composite resonance from lipid (CH2-CH2-(C═O)—O—) and methine protons of the alkyl side chain cholesterol increased by 239% (p<0.001). This was accompanied by an increase in of approximately 36% (p<0.001) in triglyceride as determined by the cross peak G″ from the triglyceride backbone.

The metabolites were all increased in the pre-menopausal high density cohort, with the composite resonances consistent with choline and or tyrosine up 900% (p<0.001); glycerophosphocholine (GPC) (glycerol moiety), glutamine/glutamate up 733% (p<0.001); ethanolamine 600% (p<0.001), the composite choline, phosphocholine 480% (p<0.001); taurine, glucose 450% (p<0.001), scyllo-inositol and glucose 420% (p<0.00): myo-inositol 350% (p<0.001), creatine, GPC, aspartate, phosphocreatine 263% (p<0.001) and the composite choline, myo-inositol 244% (<0.001) (Table 2) (FIG. 2).

### Post-Menopausal Participants

Compared to those with low density breast tissue post-menopausal women with high density breast tissue recorded an increase in cholesterol methyl (F2:0.70. F1:0.70 ppm) of 241% (p=0.019), cholesterol sterol (F2:0.40, F1:0.40 ppm) of 437% (p=0.005); the and the composite from lipid cross peak E (CH₂—CH₂—(C═O)—O—) and methine protons of the alkyl side chain cholesterol of 303% (p=0.002) cross peak D from the unsaturated acyl chain (—HC═CH₂—CH—CH═CH—), increased by 150% (p=0.005) and cross peak C (HC═CH₂—CH₂—CH₂—CH₃) by 133%. Thus, those women with high breast density who were post-menopausal recorded a significant and large increase in cholesterol, triglyceride with a concomitant increase in unsaturated fatty acyl chains. Interestingly, while the metabolites all increased, these increments were considerably smaller than those recorded for the pre-menopausal cohort (Table 3) (FIG. 2).

### Increases in Resonance Intensities of Lipids, Cholesterol and Metabolites Mobile on the MR Timescale

The relative intensities of lipids and metabolites that are mobile on the MR timescale are shown across all four categories in FIG. 3. The resonance intensity reflects the amount of the species but also the molecular motion recorded on the MR timescale. For example, triglyceride tumbles isotopically and thus generate a narrow linewidth. Cholesterol only develops a narrow-lined spectrum when mobile, which occurs in the presence of triglyceride. They are both neutral lipids and have a natural affinity.

The question arises as to which molecular species the unsaturated lipid is associated. It cannot be the phospholipids as they are not mobile on the MR timescale. Triglyceride and cholesterol are the likely candidates. A plot of the intensity of the methyl protons of the cholesterol ring with the increase in intensity of cross peak C (HC═CH₂—CH₂—CH₂—CH₃) and cross peak D (—HC═CH₂—CH—CH═CH—), are both linear (FIG. 4). No such linear correlation was recorded for triglyceride and the methyl protons of the cholesterol ring strongly suggesting that the unsaturated chains are from cholesterol ester.

We were able to obtain a significant increase in signal to noise of the spectral data, and hence a considerable increase in the number of chemicals to evaluate from breast tissue, due to a number of factors and automation of the acquisition and post processing protocols. These are summarised as follows.
1. Voxel placement on the breast and acquisition parameters were modified as discussed below. This is important in two ways. Firstly the size of the voxel was increased by 60%. Secondly the shimming was improved from linewidth at half height of 60-80 to less than 50 Hz.
2. Evaluation of the set up accuracy, prior to recording the data on the scanner, was automated to reduce introduced errors.
3. Post processing of data analysis pathway was automated removing user error.
4. Each of these processes were accompanied by a user-guide for operators a different sites.

The spectral user guide included the following.

Place the laser in the middle of the breast. Do not place voxel too superior or inferior. Avoid being too close to skin/air interface.

When first positioning the patient, center on the middle of the breast. After the first localizer, move the table (if necessary) so that the voxel is as close as possible to magnetic isocenter. This improves the quality of the shim, and thus the data.

Voxel placement should avoid the air/tissue interface in all three planes as this worsens the quality of the shim. Avoid any cystic areas, large blood vessels, the chest wall and the retro-areolar area. Where relevant, also avoid haemorrhage, masses or surgical clips. Attempt to find a halfway point between the nipple and the chest wall, without being too close to the skin's surface. Also avoid being too medial or too lateral. Double check the voxel location on a contrast-enhanced scan to ensure to avoid any region that enhances, as the presence of a large amount of Gadolinium will worsen the line shape significantly.

Where possible, position the patient with their arms down, and use cushions under the shoulders and feet. Limiting patient movement is critical, as spectroscopy adds significantly to the time the patient spends in the magnet.

Use a shim box twice the size of the voxel.

Increase the size of the shim area around the voxel. This improves the homogeneity across the voxel.

Where training exists, use the field map function in the spectroscopy tab to check the homogeneity of the magnetic field. If necessary, move the voxel and repeat the process to check for improvement. Generally, repeating the field map function two or three times improves the overall shim, as the system calculates using the previous step. Use the mode function that has the highest resolution such as prostate. It takes a little longer, but the results are generally better.

When selecting the center frequency, ensure that it is always water. Don't rely on the system for this step.

The results of the research discussed above provide a system and method for detecting breast density and thus the risk of a woman to develop breast cancer based on the breast density by looking at only the spectroscopic data without needing an MRI. By obtaining spectral data of at least one selected biochemical of the breast of a woman for whom breast density is unknown, one can compare the concentration of the at least one biochemical with reference measurements of that biochemical of women known to have low, medium and high breast density, and determine the breast density solely on the comparison.

The following Tables show the data obtained.

While a preferred embodiment of the invention has been disclosed, the invention is not limited to this embodiment.

### Tables

TABLE 1
Demographics and ADC values according to breast density subcategories

| | Breast density subcategories | | | | |
|---|---|---|---|---|---|
| | Low density | | High density | | |
| | (n = 34) | | (n = 31) | | p value |
| Age, mean (SD*) | 46.4 | (10.8) | 38.0 | (11.8) | 0.004 |
| Menopausal status, n (%) | | | | | 0.011 |
| pre-menopausal | 19 | (28) | 26 | (41) | |
| post-menopausal | 15 | (23) | 5 | (8) | |
| BMI**, mean (SD) | 28.42 | (4.52) | 22.03 | (3.67) | <0.001 |
| IBIS score, mean (SD) | 10.12 | (4.09) | 15.40 | (6.89) | 0.001 |
| ADC***, mean in mm²/s (SD) | 1348.78 | (383.72) | 580.30 | (206.45) | <0.001 |

*SD: standard deviation;
**BMI: body mass index,
***ADC: apparent diffusion coefficient

TABLE 2
Comparison of breast tissue biochemistry according to breast density adjusted for
Menopausal Status

| | | PRE-MENOPAUSAL (n = 45) | | | | POST-MENOPAUSAL (n = 20) | | | |
|---|---|---|---|---|---|---|---|---|---|
| | | | | | % change | | | | % change |
| | | | | | HIGH | | | | HIGH |
| | | Low density | High density | | DENSITY | Low density | High density | | DENSITY |
| Chemical shift | | n = 19 | n = 26 | | vs Low | n = 15 | n = 5 | | vs LOW |
| (F2, F1) ppm | Chemical species | (mean) | (mean) | p value | Density | (mean) | (mean) | p value | DENSITY |

LIPIDS

| | | | | | | | | | |
|---|---|---|---|---|---|---|---|---|---|
| 0.40, 0.40 | Cholesterol sterol | 0.00057 | 0.00117 | 0.002 | +105 | 0.00075 | 0.00403 | 0.005 | +437 |
| 0.70, 0.70 | Cholesterol methyl | 0.00907 | 0.02318 | <0.001 | +156 | 0.01296 | 0.04414 | 0.019 | +241 |
| 0.90, 0.90 | —CH₃ | 0.08617 | 0.08531 | 0.004 | −1 | 0.09047 | 0.17348 | 0.015 | +92 |
| 0.90, 1.30 | Cross peak A | 0.04289 | 0.03752 | <0.001 | −13 | 0.04137 | 0.03650 | 0.142 | −12 |
| 1.59, 1.59 | Composite of CH₂— | 0.01789 | 0.06062 | <0.001 | +239 | 0.02179 | 0.08788 | 0.002 | +303 |
| | CH₂—(C═O)—O— & | | | | | | | | |
| | methine of alkyl side | | | | | | | | |
| | chain cholesterol | | | | | | | | |
| 2.02, 2.02 | —CH₂—HC═HC— | 0.04327 | 0.04952 | <0.001 | +14 | 0.04512 | 0.10904 | 0.002 | +142 |
| 2.02, 5.31 | Cross peak C | 0.02353 | 0.02743 | 0.103 | +17 | 0.02717 | 0.06328 | 0.081 | +133 |
| 2.25, 2.25 | —CH₂—(C═O)—O— | 0.02991 | 0.03310 | 0.002 | +11 | 0.02959 | 0.06695 | 0.004 | +126 |
| 2.25, 1.59 | Cross peak F | 0.01832 | 0.02303 | <0.001 | +26 | 0.01862 | 0.03156 | 0.011 | +69 |
| 2.75, 2.75 | ═HC—CH₂—HC═ | 0.01295 | 0.01354 | 0.024 | 5 | 0.01269 | 0.03343 | 0.001 | +163 |
| 2.75, 5.31 | Cross peak D | 0.00998 | 0.01322 | 0.043 | +32 | 0.01168 | 0.02918 | 0.005 | +150 |
| 4.10, 4.10/ | —CH₂—(C═O)—O— & | 0.06470 | 0.08818 | 0.004 | +36 | 0.09963 | 0.18042 | 0.015 | +81 |
| 4.10, 4.25 | cross peak G′ | | | | | | | | |
| 4.10, 5.31 | Cross peak G | 0.01093 | 0.01157 | 0.448 | +6 | 0.02087 | 0.02149 | 0.066 | +3 |
| 4.25, 4.25 | —CH₂—O—(C═O)— | 0.04263 | 0.05106 | 0.108 | +20 | 0.04627 | 0.10414 | 0.004 | +125 |
| | (triglyceride | | | | | | | | |
| | backbone) | | | | | | | | |
| 5.31, 5.31 | —CH═HC— | 0.12266 | 0.15868 | 0.011 | +29 | 0.14544 | 0.52179 | 0.001 | +259 |

METABOLITES

| | | | | | | | | | |
|---|---|---|---|---|---|---|---|---|---|
| 3.03, 3.03 | Tyrosine, | 0.00013 | 0.00048 | <0.001 | +269 | 0.00047 | 0.00129 | 0.008 | +174 |
| | Phosphocreatine, | | | | | | | | |
| | Creatine | | | | | | | | |
| 3.12, 3.12 | Histidine | 0.00003 | 0.00019 | <0.001 | +533 | 0.00031 | 0.00040 | 0.019 | +29 |
| 3.15, 3.15 | Ethanolamine | 0.00003 | 0.00018 | <0.001 | +600 | 0.00028 | 0.00034 | 0.015 | +21 |
| 3.19, 3.19 | Choline, Tyrosine | 0.00001 | 0.00009 | <0.001 | +900 | 0.00011 | 0.00016 | 0.008 | +45 |
| 3.20, 3.20 | Choline, *PC | 0.00005 | 0.00029 | <0.001 | +480 | 0.00029 | 0.00046 | 0.033 | +59 |
| 3.25, 3.25 | Taurine, Glucose | 0.00002 | 0.00011 | <0.001 | +450 | 0.00009 | 0.00020 | 0.019 | +122 |
| 3.27, 3.27 | Myo-inositol | 0.00002 | 0.00009 | <0.001 | +350 | 0.00008 | 0.00021 | 0.015 | +163 |
| 3.35, 3.35 | Scyllo-inositol, | 0.00005 | 0.00026 | <0.001 | +420 | 0.00020 | 0.00053 | 0.011 | +165 |
| | Glucose, Histidine | | | | | | | | |
| 3.41, 3.41 | Taurine, Glucose | 0.00006 | 0.00024 | <0.001 | +300 | 0.00017 | 0.00046 | 0.033 | +171 |
| 3.50, 3.50 | Choline, Myo-inositol | 0.00009 | 0.00031 | <0.001 | +244 | 0.00022 | 0.00064 | 0.019 | +191 |
| 3.55, 3.55 | Glycerol, Myo | 0.00006 | 0.00019 | <0.001 | +217 | 0.00016 | 0.00041 | 0.005 | +156 |
| | inositol, Glycine | | | | | | | | |
| 3.61, 3.61 | Myo-inositol, **GPC | 0.00009 | 0.00035 | <0.001 | +289 | 0.00031 | 0.00066 | 0.008 | +113 |
| | (Glycerol moiety) | | | | | | | | |
| 3.64, 3.64 | Glycerol, | 0.00003 | 0.00012 | <0.001 | +300 | 0.00011 | 0.00021 | 0.042 | +91 |
| | Phosphocholine, GPC | | | | | | | | |
| | (Choline moiety) | | | | | | | | |
| 3.70, 3.70 | GPC (Glycerol | 0.00003 | 0.00025 | <0.001 | +733 | 0.00018 | 0.00030 | 0.053 | +67 |
| | moiety), Glutamine | | | | | | | | |
| 3.73, 3.73 | Glutamine, Glutamate | 0.00003 | 0.00023 | <0.001 | +667 | 0.00017 | 0.00033 | 0.098 | +94 |
| 3.78, 3.78 | Glycerol, Alanine | 0.00004 | 0.00027 | <0.001 | +575 | 0.00014 | 0.00045 | 0.042 | +221 |
| 3.90, 3.90 | Creatine, GPC | 0.00041 | 0.00149 | <0.001 | +263 | 0.00082 | 0.00278 | 0.019 | +239 |
| | (Glycerol moiety), | | | | | | | | |
| | Aspartate, | | | | | | | | |
| | Phosphocreatine | | | | | | | | |

*PC: Phosphocholine; **GPC: Glycerophosphocholine

### REFERENCES

1. McCormack, V. A. and I. dos Santos Silva, Breast Density and Parenchymal Patterns as Markers of Breast Cancer Risk: A Meta-analysis. Cancer Epidemiology Biomarkers &amp; Prevention, 2006. 15(6): p. 1159-1169.
2. Boyd, N. F., et al., Mammographic densities and breast cancer risk. Cancer Epidemiol Biomarkers Prev, 1998. 7(12): p. 1133-44.
3. Duffy, S. W., et al., Mammographic density and breast cancer risk in breast screening assessment cases and women with a family history of breast cancer. European Journal of Cancer, 2018. 88: p. 48-56.
4. Boyd, N. F., et al., Mammographic density and the risk and detection of breast cancer. N Engl J Med, 2007. 356(3): p. 227-36.
5. Veronesi, U., et al., Breast cancer. Lancet, 2005. 365(9472): p. 1727-41.
6. D'Orsi C, S. E., Mendelson E, et al., ed. ACR BI-RADS Atlas, breast imaging reporting and data system. 2013, American College of Radiology: Reston, Va.
7. Ramadan, S., et al., Lipid and Metabolite Deregulation in the Breast Tissue of Women Carrying BRCA1 and BRCA2 Genetic Mutations. Radiology, 2015. 275(3): p. 675-82.
8. Thomas, M. A., et al., Investigation of breast cancer using two-dimensional MRS. 2009. 22(1): p. 77-91.
9. Stanwell, P., et al., Neuro magnetic resonance spectroscopy using wavelet decomposition and statistical testing identifies biochemical changes in people with spinal cord injury and pain. NeuroImage, 2010. 53(2): p. 544-52.
10. Mountford, C., et al., Proton MRS Can Determine the Pathology of Human Cancers With A High Level of Accuracy. Chemical Reviews, 2004. 104: p. 3677-3704.
11. Tosh, N., et al., Two New Fucose-α (1-2)-Glycans Assigned In The Healthy Human Brain Taking The Number To Seven. Nature Scientific Reports, 2019. 9(1): p. 18806.
12. Hamilton, J. A. and J. D. Morrisett, [28] Nuclear magnetic resonance studies of lipoproteins, in Methods in Enzymology. 1986, Academic Press. p. 472-515.
13. Mountford, C. E. and L. C. Wright, Organization of lipids in the plasma membranes of malignant and stimulated cells: a new model. Trends Biochem Sci, 1988. 13(5): p. 172-7.
14. Hakumnki, J. M. and R. A. Kauppinen, <sup>1</sup>H NMR visible lipids in the life and death of cells. Trends in Biochemical Sciences, 2000. 25(8): p. 357-362.
15. Khandelia, H., et al., Triglyceride blisters in lipid bilayers: implications for lipid droplet biogenesis and the mobile lipid signal in cancer cell membranes. PLoS One, 2010. 5(9): p. e12811.
16. King, N. J. C., M. H. Ward, and K. T. Holmes, Magnetic resonance studies of murine macrophages: Proliferation is not a prerequisite for acquisition of an ‘activated’ high resolution spectrum. FEBS Letters, 1991. 287(1): p. 97-101.
17. Abrahamsson, A., et al., Dense breast tissue in postmenopausal women is associated with a pro-inflammatory microenvironment in vivo. OncoImmunology, 2016. 5(10).
18. Shi, J., et al., Cytokines and Abnormal Glucose and Lipid Metabolism. Frontiers In Endocrinology, 2019. 10.
19. Hanahan, D. and R. A. Weinberg, Hallmarks of cancer: the next generation. Cell, 2011. 144(5): p. 646-74.
20. Morris, B. A., et al., Collagen Matrix Density Drives the Metabolic Shift in Breast Cancer Cells. EBioMedicine, 2016. 13(C): p. 146-156.
21. Mavi, A., et al., The Effect of Age, Menopausal State, and Breast Density on \hat{ }sup 18\hat{ }F-FDG Uptake in Normal Glandular Breast Tissue. The Journal of Nuclear Medicine, 2010. 51(3): p. 347-52.
22. Pasta, V., et al., An association of boswellia, betaine and myo-inositol (Eumastos) in the treatment of mammographic breast density: a randomized, double-blind study. Eur Rev Med Pharmacol Sci, 2015. 19(22): p. 4419-26.
23. El Agouza, I. M., et al., Taurine: a novel tumor marker for enhanced detection of breast cancer among female patients. Angiogenesis, 2011. 14(3): p. 321-30.
24. Mulsow, J., et al., Establishing a family risk assessment clinic for breast cancer. Breast J, 2009. 15 Suppl 1: p. S33-8.
25. Tyrer, J., S. W. Duffy, and J. Cuzick, A breast cancer prediction model incorporating familial and personal risk factors. Stat Med, 2004. 23(7): p. 1111-30.
26. Mountford C, R. S., Stanwell P, Malycha P., Proton MRS of the breast in the clinical setting. NMR in biomedicine., 2009. 22(1): p. 54.
27. Ogg, R. J., R. B. Kingsley, and J. S. Taylor, WET, a T1- and B1-Insensitive Water-Suppression Method for in Vivo Localized 1H NMR Spectroscopy. Journal of Magnetic Resonance, Series B, 1994. 104(1): p. 1-10.

1. A method for enabling detection of breast density of a subject, comprising: obtaining spectral data, using a spectroscopy device, of a breast of a subject, and processing the spectral data with a processor to obtain a measurement of the concentration of at least one selected biochemical whose concentration varies with breast density, to enable a comparison of the measurement of the concentration with reference measurements of the concentration of the selected biochemical of subjects having varying known levels of breast tissue density correlated with the concentration of the selected biochemical, to enable a determination of the breast density of the subject by reference to the concentration of the selected biochemical.

2. The method according to claim 1, wherein the selected biochemical is at least one of cholesterol (sterol and methyl), triglycerides, unsaturated fatty acyl chains or at least one of selected metabolites.

3. The method according to claim 2, wherein at least one of the selected metabolites is choline, tyrosine, glycerophosphocholine, glutamine/glutamate, ethanolamine, composite choline, phosphocholine, taurine, glucose, scyllo-inositol, glucose, myo-inositol, creatine, GPC, aspartate, phosphocreatine, composite choline or myo-inositol.

4. The method according to claim 1, wherein the reference measurements are those of pre-menopausal women and post-menopausal women in separate groups, and the spectral data of the subject are compared to the reference measurements of the relevant group depending on whether the subject is pre-menopausal or post-menopausal.

5. A system for enabling detection of breast density of a subject, comprising:
a spectrometer for obtaining spectral data of a breast of a subject, and
a processor for processing the spectral data to obtain a measurement of the concentration of at least one selected biochemical whose concentration varies with breast density to enable a comparison of the measurement with reference measurements of the concentration of the selected biochemical of subjects known to have varying known levels of breast tissue density correlated with the concentration of the selected biochemical, to enable a determination of the breast density of the subject by reference to the concentration of the selected biochemical.

6. The system according to claim 5, wherein the selected biochemical is at least one of cholesterol (sterol and methyl), triglycerides, unsaturated fatty acyl chains or at least one of selected metabolites.

7. The system according to claim 6, wherein at least one of the selected metabolites is choline, tyrosine, glycerophosphocholine, glutamine/glutamate, ethanolamine, composite choline, phosphocholine, taurine, glucose, scyllo-inositol, glucose, myo-inositol, creatine, GPC, aspartate, phosphocreatine, composite choline or myo-inositol.

8. The system according to claim 5, wherein the reference measurements are those of pre-menopausal women and post-menopausal women in separate groups, and the spectral data of the subject are compared to the reference measurements of the relevant group depending on whether the subject is pre-menopausal or post-menopausal.

9. A method of making a breast density detection system for enabling a determination of breast density of a subject using the concentration of at least one selected biochemical in the subject's breast tissue whose concentration varies with breast density, comprising:
using a magnetic resonance imaging device to obtain magnetic resonance images of a plurality of breasts of women having different breast densities;
using a spectrometer to obtain spectral data from the plurality of the women's breasts to obtain the concentration of at least one selected biochemical in the plurality of the breasts, wherein the concentration of the selected bio-chemical varies with the breast density; and
using a processor to correlate the breast density with the concentration of the selected biochemical to obtain a reference system of reference measurements which correlates breast density with the concentration of the selected biochemical, whereby the breast density of a subject can be determined by obtaining the spectral data and the concentration of the selected biochemical.

10. The method according to claim 9, wherein the selected biochemical is at least one of cholesterol (sterol and methyl), triglycerides, unsaturated fatty ackyl chains or at least one of selected metabolites.

11. The method according to claim 9, wherein at least one of the selected metabolites is choline, tyrosine, glycerophosphocholine, glutamine/glutamate, ethanolamine, composite choline, phosphocholine, taurine, glucose, scyllo-inositol, glucose, myo-inositol, creatine, GPC, aspartate, phosphocreatine, composite choline or myo-inositol.

12. The method according to claim 9, wherein the reference measurements are those of pre-menopausal women and post-menopausal women in separate groups, whereby the spectral data of the subject are compared to the reference measurements of the relevant group depending on whether the subject is pre-menopausal or post-menopausal.

13. A method of using a breast density detection system to determine the breast density of the subject, the system having been obtained by:
using a magnetic resonance imaging device to obtain magnetic resonance images of a plurality of breasts of women having different breast densities;
using a spectrometer to obtain spectral data from the plurality of breasts to obtain the concentration of at least one selected biochemical in the plurality of the breasts which concentration varies with the breast density; and
using a processor to correlate the breast density with the concentration of the selected biochemical to obtain a reference system of reference measurements which correlates breast density with the concentration of the selected biochemical,
wherein the method of using comprises obtaining spectral data of the subject's breast with a spectrometer, and using a processor to determine the concentration of the selected biochemical, and to determine the breast density by reference to the breast density which correlates with the concentration of the selected biochemical.

14. The method according to claim 13, wherein the selected biochemical is at least one of cholesterol (sterol and methyl), triglycerides, unsaturated fatty ackyl chains or at least one of selected metabolites.

15. The method according to claim 14, wherein at least one of the selected metabolites is choline, tyrosine, glycerophosphocholine, glutamine/glutamate, ethanolamine, composite choline, phosphocholine, taurine, glucose, scyllo-inositol, glucose, myo-inositol, creatine, GPC, aspartate, phosphocreatine, composite choline or myo-inositol.

16. The method according to claim 13, wherein the reference measurements are those of pre-menopausal women and post-menopausal women in separate groups, whereby the spectral data of the subject are compared to the reference measurements of the relevant group depending on whether the subject is pre-menopausal or post-menopausal.
